(19) United States
(12) Patent Application Publication
(10) Pub. No.: US 2026/0256952 A1
(43) Pub. Date: Sep. 3, 2026

(54) DUAL-MODAL TUMOR IMAGING USING FUNCTIONALIZED IRON-OXIDE NANOPARTICLES FOR MRI AND FLUORESCENCE IMAGING

(71) Applicant: University of South Florida, Tampa, FL (US)

(72) Inventors: Kevin Secades, Tampa, FL (US); Diane Allen-Gipson, Tampa, FL (US)

(21) Appl. No.: 19/552,304

(22) Filed: Feb. 27, 2026

Related U.S. Application Data
(60) Provisional application No. 63/764,608, filed on Feb. 28, 2025.

(51) Int. Cl. A61K 49/18; A61K 49/00

(52) U.S. Cl. CPC A61K 49/186; A61K 49/0052; A61K 49/0054; A61K 49/0093; A61K 2123/00

(57) ABSTRACT

Disclosed herein are systems and methods for facilitating tumor discovery without the use of radioisotopes.

### CROSS-REFERENCE TO RELATED APPLICATIONS

This application claims priority to and the filing benefit of U.S. Provisional Patent Application No. 63/764,608, filed 28 February 2025, which is incorporated by reference herein in its entirety.

### FIELD

This disclosure relates generally to tumor discovery.

### BACKGROUND

Accurate tumor imaging is essential for early cancer detection and treatment monitoring. Technetium-99m (Tc-99m) is a commonly used radioisotope for medical imaging, but relies on gamma-ray emission for the imaging. Both the gamma-ray emission and radioisotope presence pose challenges to safety, accessibility, and regulatory constraints.

What is needed are systems and methods of tumor cell discovery that are safe for a patient, easily produced, environmentally friendly, exhibit reasonable shelf-lives, and lower cost.

### SUMMARY OF THE INVENTION

Covered embodiments of the invention are defined by the claims, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings, and each claim.

Disclosed herein is a method for providing a tumor discovering compound that can include synthesizing iron oxide (Fe₃O₄) nanoparticles (IONPs), coating the IONPs with a poly(ethylene glycol) (PEG) layer, wherein the PEG layer includes a folic acid component (PEG-FA) to provide a PEG-FA coated IONP (PEG-FA-IONP), purifying the PEG-FA-IONPs, labeling the PEG-FA-IONPs with a fluorescent chromophore to provide a PEG-FA-dye-IONP, and purifying the PEG-FA-dye-IONPs.

In some embodiments, synthesizing the IONPs can include preparing an alkaline solution in a first reaction vessel, preparing an iron salt precursor solution in a second reaction vessel, maintaining the iron salt precursor solution at a temperature of from 60 °C to 100 °C, a pH of from 8 to 10, and stirring at a rate of from 300 revolutions per minute (RPM) to 400 RPM in the second reaction vessel, and adding the alkaline solution dropwise to the iron salt precursor solution in the second reaction vessel. In other embodiments, coating the IONPs with the PEG-FA layer can include preparing the PEG-FA solution in a third reaction vessel, maintaining the iron salt precursor solution at a temperature of from 60 °C to 100 °C, a pH of from 8 to 10, and stirring at a rate of from 300 revolutions per minute (RPM) to 400 RPM in the second reaction vessel, and adding the PEG-FA solution dropwise to the iron salt precursor solution in the second reaction vessel. In further embodiments, purifying the PEG-FA-IONPs can include magnetically separating the PEG-FA-IONPs from a reaction medium comprising unreacted salts or excess reagents, washing the magnetically separated PEG-FA-IONPs, and drying the PEG-FA-IONPs. In some embodiments, labeling the PEG-FA-IONPs with a fluorescent chromophore to provide the PEG-FA-dye-IONPs can include adding the PEG-FA-IONPs to a phosphate-buffered saline (PBS) solution, adding an ester-functionalized dye solution to the PEG-FA-IONPs/PBS solution to provide a dye-PEG-FA-IONPs solution, and incubating the dye-PEG-FA-IONPs solution to provide the PEG-FA-dye-IONPs. In other embodiments, purifying the PEG-FA-dye-IONPs can include magnetically separating the PEG-FA-dye-IONPs from a supernatant, washing the separated PEG-FA-dye-IONPs in a PBS solution, and washing the separated PEG-FA-dye-IONPs in deionized water.

Also disclosed herein is a method of identifying cancerous cells in a body that can include introducing a fluorescent chromophore-labeled-cancer-specific-ligand-functionalized nanoparticle into the body, binding the fluorescent chromophore-labeled-cancer-specific-ligand-functionalized nanoparticle to a cell overexpressing folate receptors, and imaging the cells overexpressing folate receptors having the fluorescent chromophore-labeled-cancer-specific-ligand-functionalized nanoparticle bound thereon, wherein imaging comprises contrast-enhanced magnetic resonance imaging (MRI), optical imaging, or a combination thereof to specify a tumor cell.

In some embodiments, the nanoparticle can be an iron oxide (Fe₃O₄) nanoparticle (IONP), the cancer-specific ligand can be folic acid, and the fluorescent chromophore can be a near-infrared (NIR) light emitting dye or a visible light emitting dye. For example, the fluorescent chromophore-labeled-cancer-specific-ligand-functionalized nanoparticle can be a fluorescent dye-labeled, poly(ethylene glycol)-folic acid (PEG-FA) coated iron oxide nanoparticle (PEG-FA-dye-IONP). In further embodiments, introducing the fluorescent chromophore-labeled-cancer-specific-ligand-functionalized nanoparticle into the body comprises injecting a dispersion of the PEG-FA-dye-IONPs into the body. In other embodiments, binding the PEG-FA-dye-IONPs to a cell overexpressing folate receptors comprises urging the PEG-FA-dye-IONPs toward a cell, imaging the PEG-FA-dye-IONPs provides a T₂-weighted MRI contrast indicated by dark areas in an MRI image and optical imaging, the optical imaging including exciting the fluorescent chromophore to provide an optical image of the PEG-FA-dye-IONPs bound to the cell overexpressing folate receptors. For example, the fluorescent chromophore can have an excitement wavelength (λ) from 590 nanometers (nm) to 675 nm, an emission wavelength from 650 nm to 750 nm, and a full-width half-maximum (FWHM) from 5 nm to 50 nm. In certain embodiments, combining contrast-enhanced MRI and optical imaging provides an in-vitro and in-vivo safe cancer-cell identifying system.

Further disclosed herein is a dual-modal tumor imaging system that can include an iron oxide (Fe₃O₄) nanoparticle (IONP), a poly(ethylene glycol) (PEG) layer disposed on the IONP, wherein the PEG layer can include a folic acid component (PEG-FA), and a fluorescent chromophore conjugated onto a portion of the folic acid component. In some embodiments, the fluorescent chromophore comprises an excitement wavelength (λ) from 590 nanometers (nm) to 675 nm, an emission wavelength from 650 nm to 750 nm, and a full-width half-maximum (FWHM) from 5 nm to 50 nm.

### BRIEF DESCRIPTION OF THE DRAWINGS

The accompanying drawings, which are incorporated in and constitute a part of this specification, illustrate possible embodiments of the disclosure and together with the description, serve to explain the principles of the disclosure.

FIG. 1 is a flowchart illustrating a method of providing a tumor cell identifying system according to some embodiments of the present disclosure.

FIG. 2 is a flowchart illustrating a method of administering a tumor cell identifying system according to some embodiments of the present disclosure.

FIG. 3 illustrates a tumor cell identifying system according to some embodiments of the present disclosure.

FIG. 4 is a digital image showing a tumor cell identifying system according to some embodiments of the present disclosure.

FIG. 5 is a graph showing a spectroscopic standard curve according to some embodiments of the present disclosure.

FIG. 6 is a graph showing a spectroscopic calibration curve according to some embodiments of the present disclosure.

FIG. 7 is a graph showing a spectroscopic standard curve according to some embodiments of the present disclosure.

FIGS. 8 and 9 are microscopy images showing a tumor cell identifying system according to some embodiments of the present disclosure.

FIG. 10 is a graph showing peak-to-peak line analysis of a tumor cell identifying system according to some embodiments of the present disclosure.

FIGS. 11 and 12 are graphs showing cell uptake of a tumor cell identifying system according to some embodiments of the present disclosure

### DETAILED DESCRIPTION

Disclosed herein are systems and methods to provide a dual-modal imaging platform achieved by functionalizing iron-oxide nanoparticles (IONPs) with a fluorescent chromophore (e.g., Cy5), enabling both contrast-enhanced magnetic resonance imaging (MRI) and optical imaging for improved tumor specificity (e.g., cancer cells, cancerous cells, tumor cells, or the like). IONPs provide a strong T₂-weighted MRI contrast. A conjugated near-infrared (NIR) light emitting chromophores can provide high-resolution fluorescence imaging. Combing the contrast-enhanced MRI and fluorescence imaging can provide a system adaptable for both in-vitro and in-vivo applications. Additionally, the IONPs can be functionalized with cancer-specific ligands, e.g., folic acid (FA), thus providing a targeted delivery of the nanoparticle (NP) system to tumor cells that overexpress folate receptors (FRs). The systems and methods described herein, e.g., the multi-modal imaging platform, can provide a precise, non-radioactive, cost-effective alternative to nuclear medicine tracers.

The present invention may be understood more readily by reference to the following detailed description of preferred embodiments of the invention and the Examples included therein and to the Figures and their previous and following description.

As used herein, the terms “invention,” “the invention,” “this invention,” and “the present invention” are intended to refer broadly to all of the subject matter of this patent application and the claims below. Statements containing these terms should be understood not to limit the subject matter described herein or to limit the meaning or scope of the patent claims below.

As used herein, the meaning of “a,” “an,” and “the” includes singular and plural references unless the context clearly dictates otherwise.

All ranges disclosed herein are to be understood to encompass any and all endpoints as well as any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10.

As used herein, the meaning of “room temperature” can include a temperature of from about 15° C to about 30° C, for example about 15° C, about 16° C, about 17° C, about 18° C, about 19° C, about 20° C, about 21° C, about 22° C, about 23° C, about 24° C, about 25° C, about 26° C, about 27° C, about 28° C, about 29° C, or about 30° C.

The term “and/or” when used in a list of two or more items, means that any one of the listed items can be employed by itself or in combination with any one or more of the listed items. For example, the expression “A and/or B” is intended to mean either or both of A and B, i.e., A alone, B alone, or A and B in combination. The expression “A, B and/or C” is intended to mean A alone, B alone, C alone, A and B in combination, A and C in combination, B and C in combination or A, B, and C in combination.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Reference will now be made in detail to the present embodiments of the invention, examples of which are illustrated in the accompanying drawings. Wherever possible, the same reference numbers are used throughout the drawings to refer to the same or like parts.

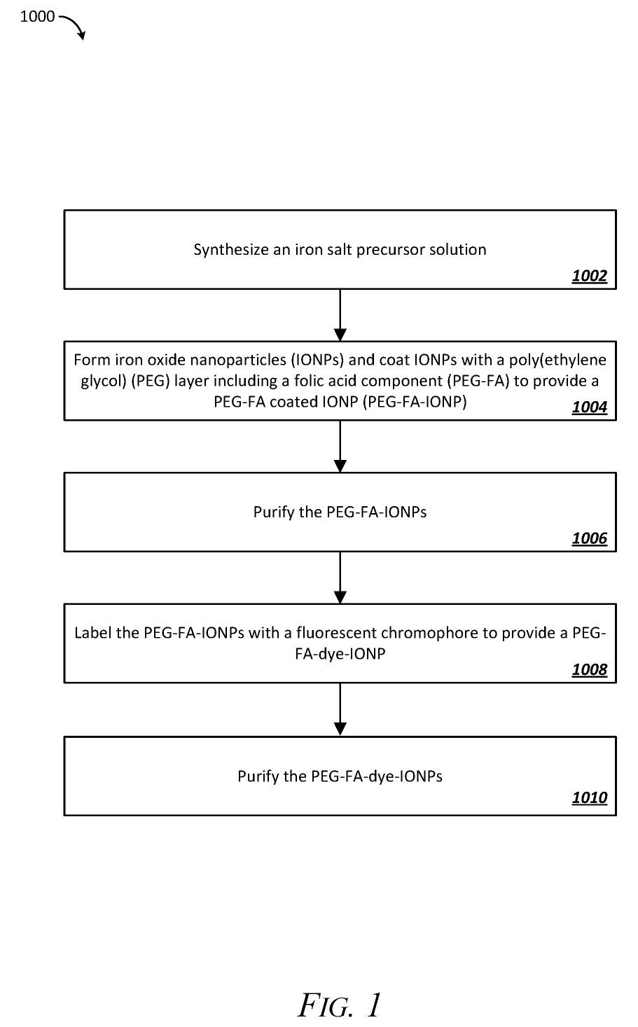

FIGS. 1-3 exemplify systems and methods for providing and using safe, low-cost, and effective materials for identifying and marking cancer cells in a body. In other words, the systems and methods described herein can find, bind, and trace cancer cells without the use of radioisotopes, gamma radiation, and high-cost synthetic methods. Accordingly, the systems and methods described herein provide a more efficient and readily available way to discover cancerous cells in a patient’s body.

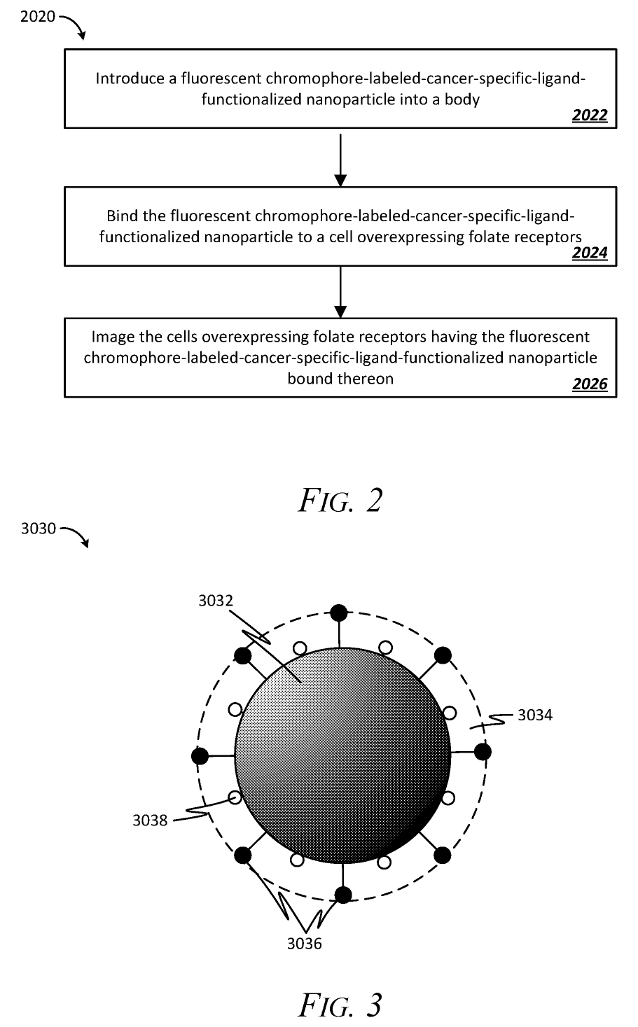

In some embodiments, a method of preparing and using a functionalized nanoparticle is illustrated in FIGS. 1 and 2. Referring now to FIG. 1, a method 1000 of preparing functionalized nanoparticles 3030 (see FIG. 3) according to embodiments described herein is illustrated. For example, at operation 1002, iron oxide (Fe₃O₄) nanoparticles (IONPs) can be synthesized. In some embodiments, IONPs can be synthesized via a modified one-pot co-precipitation method. The synthesis operation 1002 can include preparation of an alkaline solution, formation of an iron precursor solution, and a co-precipitation reaction.

### Iron Salt Precursor and IONP Synthesis Operation 1002

### Alkaline Solution Preparation

To initiate the synthesis operation 1002, an alkaline solution can be prepared in a first reaction vessel (e.g., a beaker, a round-bottom flask, an Erlenmeyer flask, or any suitable reaction vessel) by dissolving sodium hydroxide (NaOH) pellets in deionized (DI) water at a concentration of from about 60 milligrams per milliliter (mg/mL) to about 100 mg/mL (e.g., the NaOH concentration in DI water can range from about 60 mg/mL to about 99 mg/mL, from about 61 mg/mL to about 100 mg/mL, from about 65 mg/mL to about 95 mg/mL, from about 70 mg/mL to about 90 mg/mL, from about 75 mg/mL to about 85 mg/mL, or about 80 mg/mL). The mixture can then be heated to a temperature from about 60° C to about 100° C (e.g., from about 61° C to about 100° C, from about 60° C to about 99° C, from about 65° C to about 95° C, from about 70° C to about 90° C, from about 75° C to about 85° C, or about 80° C). The mixture can be stirred (e.g., using a magnetic stir bar, an impeller, or the like) at a rate of about from 300 revolutions per minute (RPM) to about 400 RPM (e.g., from about 325 RPM to about 375 RPM) until the NaOH pellets are dissolved, providing a 2 molar (M) alkaline solution.

### Iron Salt Precursor Solution Preparation

The synthesis operation 1002 can continue by heating about 100 mL DI water to about 80 °C in a second reaction vessel. Ferric chloride (FeCl₃ 0.54 g) and ferrous sulfate heptahydrate (FeSO₄∙7H₂O 0.46 g) can be dissolved in the heated DI water to provide an iron salt precursor solution.

### Iron Oxide Nanoparticles (IONPs) Co-precipitation

In some embodiments, the synthesis operation 1002 can continue by transferring the alkaline solution to a dropping funnel and adding the alkaline solution dropwise to the second reaction vessel containing the iron salt precursor solution. The reaction can be performed at a temperature of about 80 °C, stirring at 350 RPM, and maintaining a pH of from about 8 to about 10 throughout the dropwise addition of the alkaline solution. An IONP dispersion can form rapidly via the alkaline solution precipitating into the iron salt precursor solution.

### IONP Forming and Coating Operation 1004

In some embodiments, synthesizing and coating the IONPs can be performed in a single operation. To provide and coat the IONPs, at the coating operation 1004, poly(ethylene glycol)-Folic Acid-Iron Oxide Nanoparticles (PEG-FA-IONPs) can be provided via another modified one-pot co-precipitation method. Coating the IONPs can include preparing a PEG-FA solution, adding the PEG-FA solution to the IONP dispersion, co-precipitating the PEG-FA solution and IONP dispersion, and magnetically purifying the coated PEG-FA-IONPs.

### Preparation of PEG-Folic Acid Coating Solution

In coating operation 1004, a coating solution can be prepared by dissolving NaOH in DI water at a concentration of about 80 mg/mL in a third reaction vessel, providing the 2 M alkaline solution. Eight-thousand molecular weight (M_{w}) PEG (PEG-8000) and FA in a ratio of about 100:1 by weight (w/w) can be added to the 2 M NaOH solution. In some embodiments, the PEG-8000/FA-2 M NaOH mixture can heated to about 80 °C and stirred until the PEG-8000/FA is fully dissolved in the 2 M MaOH solution.

### Coated Iron Oxide Nanoparticles Co-precipitation

Continuing operation 1004, the PEG-FA coating solution can be transferred to the iron salt precursor solution via dropwise addition while maintaining the reaction temperature at about 80° C and a pH of about 10. The mixture can be stirred at 350 RPM throughout the PEG-FA coating solution addition. In some embodiments, in coating operation 1004, IONPs can rapidly form and almost immediately be coated with a FA-containing PEG layer (e.g., a PEG-FA shell having an IONP core) during a nucleation phase. Not to be bound by theory, supersaturated Fe²⁺ and Fe³⁺ species can rapidly assemble into Fe₃O₄ clusters, providing IONP nucleation sites (e.g., particle seeds). As the IONPs grow from the nucleation sites, the PEG-FA shell can form around the IONPs.

### Coated IONPs Purification Operation 1006

In some embodiments, at operation 1006, the coated IONPs can be purified to remove any unreacted salts and/or excess reagents remaining after the coating operation 1004. Magnetic separation can be employed to isolate the coated IONPs from the reaction medium (e.g., the supernatant) due to the magnetic properties of the IONPs. In some embodiments, the washing can be repeated to ensure that a final dispersion contains only coated IONPs.

### Magnetically Separating and Washing

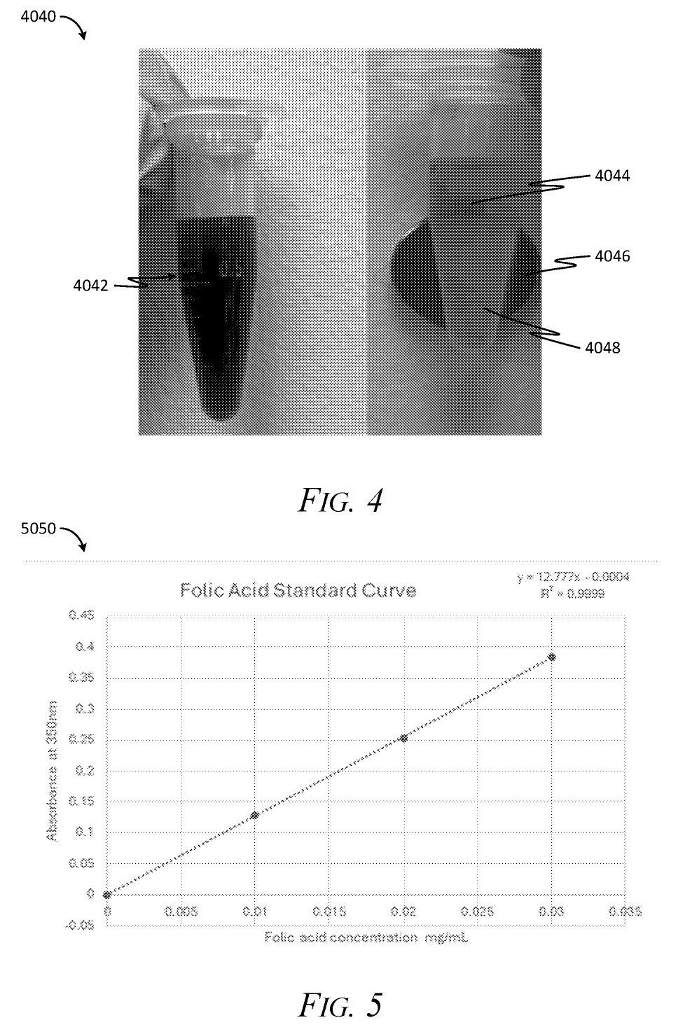

Following the forming and coating operation 1004, the IONP dispersion can be exposed to an external permanent magnet for about 15 minutes to isolate the magnetic IONPs. FIG. 4 is a digital image 4040 showing a uniformly dispersed IONP dispersion 4042 precipitating out of suspension and forming an IONP cluster 4044 upon exposure to the external permanent magnet 4046. The supernatant 4048 can be removed without disturbing the magnetized coated IONPs. The magnetically separated coated IONPs can be repeatedly washed with DI water until the solution reaches a neutral pH of about 7. Upon achieving a neutral pH, the suspension can be subjected to a final magnetic separation for about 15 minutes, and the remaining washing supernatant can be removed, providing a magnetic ferrofluid.

### IONP Drying

The reaction vessel containing the magnetic ferrofluid can be transferred to a drying oven and dried at about 60 °C for about 24 hours to provide a dry IONP powder. After about 24 hours, the reaction vessel can be removed from the drying oven and the dried IONPs can be transferred to a storage vessel (e.g., a conical tube, a glass vial, or the like). The dried IONPs can be stored in a desiccator at a temperature from about 1 °C to about 10 °C (e.g., about 1.5 °C to about 10 °C, from about 2 °C to about 9 °C, from about 3 °C to about 8 °C, or about 4 °C). In some embodiments, storage temperature can serve to preserve a colloidal stability, surface chemistry, and magnetic performance of the IONPs over time. For example, temperature-dependent processes may degrade the quality of the IONPs, particularly when stored at room temperature. At room temperature, IONPs can undergo oxidation or phase transitions (e.g., magnetite → maghemite) when stored at temperatures greater than about 10 °C. Cooler storage helps maintain consistent magnetic properties and batch-to-batch reproducibility. In addition, any surface functionalized groups (e.g., the fluorescent chromophore, folic acid, and/or PEG) may be sensitive to thermal degradation. Cooler storage reduces the rate of ligand and chemical breakdown. Storage in a desiccator at low temperature limits rehydration of the dried particles, as hydration can promote oxidation.

### Fluorescent Chromophore PEG-FA-IONPs Labeling Operation 1008

In some embodiments, at operation 1008 the coated PEG-FA-IONPs can be performed using a 1-ethyl-3-(3-dimethylaminopropyl)carbodiimide – N-hydroxysuccinimide (EDC/NHS) conjugation reaction. In some embodiments, the chromophore can be a near infrared (NIR) emitting chromophore having an excitation wavelength (λ) ranging from about 590 nanometers (nm) to about 675 nm, an emission wavelength ranging from about 650 nm to about 750 nm, and a full-width half-maximum (FWHM) emission spectrum ranging from about 5 nm to about 50 nm. For example, a Cy5 organic dye can be employed as the fluorescent chromophore. For example, a sulfo-Cy5-NHS ester (e.g., an ester-functionalized dye solution) can be used such that the NHS ester can react with primary amines present on the FA in the PEG-FA shell of the Purify the PEG-FA-IONPs.

### Fluorescent Chromophore Conjugation to PEG-FA-IONPs

In some embodiments, at operation 1008, up to a 33:1 molar ratio of fluorescent chromophore to FA can be used for the EDC/NHS conjugation reaction to achieve efficient NHS-ester linking, resulting in a dye-labeled PEG-FA-IONP (e.g., PEG-FA-dye-IONPs). For example, the molar ratio of Cy5 to FA can be about 2:1, about 3:1, about 4:1, about 5:1, about 6:1, about 7:1, about 8:1, about 9:1, about 10:1, about 15:1, about 20:1, about 25:1, about 30:1, about 31:1, about 32:1, or about 33:1. As such, a large molar excess for the fluorescent chromophore conjugation via NHS-ester conjugation can drive the reaction to completion while ensuring efficient labeling of the available FA groups. Fluorescent chromophore labeling can be scalable depending on a quantity required for accurate characterization readings, with reagent volumes adjusted proportionally to maintain the 2:1 molar ratio. In some embodiments, the fluorescent chromophore can be any one of a visible light emitting dye, a near-infrared (NIR) light emitting dye, or a combination thereof. For example, 1 mL PBS can be used as the reaction medium. PEG-FA-IONPs can be added to the PBS at a concentration of 1 mg/mL to provide a PEG-FA-IONPs/PBS solution, followed by adding 1 mg of the sulfo-Cy5-NHS ester to provide a dye-PEG-FA-IONP solution. The dye-PEG-FA-IONP solution can be mixed by pipetting. Following mixing, the dye-PEG-FA-IONP solution can be incubated in a dark environment for about 1.5 hours to allow attachment of the fluorescent chromophore via the EDC/NHS reaction to provide a chromophore-labeled-cancer-specific-ligand-functionalized nanoparticle (PEG-FA-dye-IONP).

### Fluorescent Chromophore-Labeled IONP Purification Operation 1010

Following attachment of the fluorescent chromophore, at operation 1010 the mixture can be magnetically separated and the supernatant can be removed. The IONP cluster 4044 (FIG. 4) can be washed with 1 mL PBS to remove excess unbound dye. The washing can repeated two additional times or as needed. The nanoparticles can then be washed with DI water to remove any residual PBS. The final PEG-FA-dye-IONP mixture can be stored in 1 mL HPLC grade ultrapure water at about 4 °C.

Referring now to FIG. 2, a method 2020 of administering the functionalized nanoparticles 3030 (see FIG. 3) according to embodiments described herein is illustrated. For example, at operation 2022, the PEG-FA-dye-IONPs can be introduced into the body of a patient or a cell culture prepared from suspect cells extracted from a body. At operation 2024, the FA component of the PEG-FA-dye-IONPs can bind to folate receptors being overexpressed by cancerous cells. At operation 2026, cells can be imaged via contrast-enhanced MRI and/or optical fluorescence imaging to identify cells bound with the PEG-FA-dye-IONPs.

At operation 2022, the PEG-FA-dye-IONPs can be dispersed in an aqueous medium (e.g., water, distilled water, purified water, or the like). The aqueously-dispersed PEG-FA-dye-IONPs can then be urged into a body by targeted injection, e.g., intravenous injection, muscular injection, tissue injection, or the like.

Once introduced to the body, at operation 2024 the PEG-FA-dye-IONPs can selectively bind to cells that are overexpressing folate receptors as is typical of cancerous cells. In some embodiments, the FA component of the PEG shell coating the IONPs can seek and bind to the overexpressed folate receptors, thus marking the cancerous and/or potentially cancerous cells.

Following introduction into the body and attachment of the PEG-FA-dye-IONPs to the targeted cells, imaging can be performed to determine if the PEG-FA-dye-IONPs are bound to any cells overexpressing folate receptors. In some embodiments, contrast-enhanced MRI can be used as the IONPs exhibit a strong T₂-weighted MRI contrast, displaying as dark areas in a contrast-enhanced MRI scan. Additionally, optical imaging, e.g., fluorescence spectroscopy, can be used to visually image the PEG-FA-dye-IONPs bound to cells overexpressing the folate receptors via the fluorescent chromophore (e.g., the Cy5 organic dye conjugated to the PEG-FA-IONPs. In some embodiments, contrast-enhanced MRI and optical imaging can be used in tandem to provide high-accurate and highly-precise identification of cells overexpressing folate receptors, e.g., cancerous cells.

FIG. 3 is an illustration of the PEG-FA-dye-IONPs 3030 according to embodiments described herein. The PEG-FA-dye-IONPs 3030 can have a core-shell architecture including the iron oxide nanoparticle (IONP) 3032 coated with the poly(ethylene glycol) (PEG) shell 3034. As described previously, the PEG shell 3034 can include a folic acid (FA) component 3036. Additionally, the PEG-FA-dye-IONPs 3030 can include a fluorescent chromophore 3038 (e.g., the Cy5 organic dye).

As described previously, the IONP 3032 can provide a strong T₂-weighted MRI contrast during contrast-enhanced MRI imaging. The fluorescent chromophore 3038 can provide optical imaging and evaluation of the PEG-FA-dye-IONPs 3030. The FA component 3036 can seek overexpressed folate receptors and bind thereto, effectively marking cells that are overexpressing the folate receptors.

In some embodiments, the PEG-FA-dye-IONPs 3030 can be stored, transported, and administered via an aqueous medium. In some embodiments, the PEG-FA-dye-IONPs 3030 can be stored in water for at least 28 days, which is a vast improvement over the shelf-life of typical cancer-tracing compounds that typically have a shelf-life of up to about 6 hours. The PEG-FA-dye-IONPs 3030 can exhibit a very low cytotoxicity, which is immensely safer than using radioisotopes and gamma radiation to image cancerous cells. Moreover, the simple, one-pot type synthetic route to provide the PEG-FA-dye-IONPs 3030 is more cost-effective, environmentally sound, and safe when compared to radioisotope preparation.

### Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, compositions are indicated in parts by weight, temperature is in °C or is at ambient temperature, and pressure is at or near atmospheric pressure.

### Materials

Chemicals and Reagents: 97% Iron (III) chloride, 99% Iron (II) sulfate heptahydrate, 97% Sodium hydroxide pellets, 97% Folic acid, Poly (ethylene glycol)-8000, and pH 7.2 sterile-filtered Phosphate buffered saline were purchased from Sigma Aldrich. Sulfo-Cyanine5 NHS ester was purchased from Lumiprobe. HPLC grade ultrapure water was purchased from Alfa Aesar. MTT Assay Kit purchased from BioVision.

Cell Lines: NuLi-1 (human immortalized bronchial epithelial) and HCC4006 (human immortalized lung adenocarcinoma) and A549 (human immortalized epithelial lung cancer) cells were purchased from ATTC . Nuli-1 cells were cultured and propagation maintained using BEGM media supplemented with BEGM additives and 5% penicillin/streptomycin (Lonza Pharmaceuticals). HCC4006 cells were cultured and propagation maintained using RPMI-1640 media (ATCC) supplemented with 10% fetal bovine serum (FBS) and 5% penicillin-streptomycin. A549 Cells were cultured and propagated maintained using DMEM-F12 media (Fisher Scientific) with 10% FBS and 5% penicillin/streptomycin. Trypsin EDTA 1x, 0.05% Trypsin/0.53 mM EDTA in HBSS was purchased from Fisher, Fetal Bovine Serum from Millipore Sigma-Aldrich, Penicillin-Streptomycin from Gibco.

Equipment: Laboratory instruments including glassware, hot plates, scissor jack stand, funnel support stands, 1.5mL microcentrifuge tubes, 50mL conical tubes, glass cuvette, Malvern Panalytical Folded Capillary Zeta Cell, desiccator, magnetic stir bars, funnels, plate reader, pH paper, oven, zeta potential analyzer (Malvern Zetasizer Nano ZS from Malvern Instruments), JEOL 1400 transmission electron microscope (TEM), ultrasonic bath sonicator (Thermo-Fisher Scientific), vortex mixer, Eppendorf pipettes, motorized pipette, and 24 and 96 well plates with lid (CytoOne) and EVOS Cell imaging microscope ( Thermo-Fisher Scientific).

### In-vitro Evaluation

Cell Lines and Culture Media for Biological Experiments:NuLi-1, HCC4006 and A549 cells were used for biological experiments to confirm cytotoxicity, and uptake of the IONPs, CY5 and PEG-FA-Cy5-IONPs. All cells were cultured and maintained as previously described. For all biological experimental studies, all cells were placed in serum-free media to avoid any interactions between the chemical reagents in the MTT or cellular uptake studies. Briefly, the experimental media consists of Complete BEGM media which is serum-free and was used for both Nuli-1 and HCC4006 cell lines. The A549 cells used Complete DMEM-F12 minus 10 % FBS (serum-free). Cells were cultured and maintained in incubator at 37 °C, 5 % CO₂.

Cell Culture Conditions: All cell lines were maintained under standard incubator conditions at 37 °C with 5 % CO₂. Prior to trypsinization, all serum-contain media was removed, and cells were rinsed with PBS to prevent FBS-mediated inhibition of trypsin activity. Cells were detached using trypsin as described by manufacture, centrifuged, and cell counted via hemocytometer and plated on 96 well plates with their respective experimental media.

Cell Seeding for Uptake and Cytotoxicity studies: All cytotoxicity cell experiments were performed on 96 well plates with n=3 independent trials for each cell line. In trial 1, NuLI-1 and HCC4006 cells were seeded at 10,000 cells per well (1x10⁴ cells/well) in 200 µL of serum-free medium. Based on the low metabolic signal observed in Trial 1, the NuLi-1 seeding density was increased to 20,000 cells per well (2x10⁴ cells/well) for Trial 2, while HCC4006 remained at 10,000 cells per well. Trial 3 was performed using the same seeding densities as Trial 2 to ensure consistency and reproducibility. Uptake studies for NuLi-1 and HCC4006 cells utilized the same 20,000 cells per well (2x10⁴ cells/well) density. Cells were treated for 24 hours with IONPs, Cy5 and IONPs-Cy5 at various concentrations ranging from (1.25 µg/mL, 2.5 µg/mL, 5 µg/mL, 10 µg/mL, and 25 µg/mL).

MTT Cytotoxicity Assay: Cytotoxicity was evaluated to perform a standard MTT assay in 96 well plates. Prior to treatment, all cells containing serum were washed with PBS to remove residual serum, and all nanoparticle treatments utilized serum-free media to prevent interaction with the MTT reagent. The MTT kit consists of two reagents, Reagent A and Reagent B. Reagent A is sensitive to serum and therefore, it was necessary to remove the serum from the cells. Reagent B provides the reaction that generates the crystalized formation and purple color.

Experiments included three treatment conditions: (1) PEG-FA-Cy5-IONPs, (2) IONP-only controls, and (3) Cy5-only controls. Each were diluted to a 0.2 mg/mL working stock by combining 80 µL of 1 mg/mL stock with 320 µL of serum-free media. Cells were exposed to final concentrations of 1.25 µg/mL, 2.5 µg/mL, 5 µg/mL, 10 µg/mL, and 25 µg/mL. For each treatment condition, master mixes corresponding to these final concentrations were prepared to a total volume of 880 µL, providing sufficient volume for quadruplets to be performed for each concentration.

For each treatment conditions, a blank (no cells, IONPs, or Cy5) were prepared using the 25 µL equivalent concentration to account for any associated background absorbance at the highest dose. Each well was treated with 200 µL of the appropriate master mix and incubated for 24 hours at 37 °C, 4.8 % CO₂.

After the 24-hour treatment period, media was removed and each well received 50 µL of serum-free media and 50 µL of MTT reagent A. Plates were incubated for 3 hours at 37 °C and 4.8 % CO₂ to allow for formazan crystals to form.

Following Incubation, 150 µL of MTT reagent B, the solvent, was added to each well. Plates were wrapped in aluminum foil to avoid light exposure/activation to the Cy5 and shaken for 15 minutes to fully dissolve the formazan crystals. Absorbance was read in a plate reader at 590 nm.

Nanoparticle Uptake Assay: Nanoparticle uptake was analyzed through FA absorbance at 350 nm which serves as an indicator of folate receptors-mediated targeted IONPs delivery. Cy5 fluorescent intensity was also measured to assess nanoparticle internalization. All uptake experiments were performed in 96 well plates. Cells were seeded at the densities described previously and allowed to attach overnight. Prior to treatment, all cells containing serum were washed with PBS to remove residual serum, and all treatments were conducted in 200 µL of serum-free media.

PEG-FA-Cy5-IONPs and Cy5-only controls were each diluted to a 0.2 mg/mL working stock by combining 80 µL of 1 mg/mL stock with 320 µL of serum-free media. Cells were exposed to final concentrations of 1.25 µg/mL, 2.5 µg/mL, 5 µg/mL, and 10 µg/mL. For each treatment category, master mixes corresponding to these final concentrations were prepared to a total volume of 880 µL, providing sufficient volume for quadruplets to be performed for each concentration. Cells were treated with 200 µL of the appropriate master mix and incubated at 37 °C, 4.8 % CO₂ based on experimental time parameters.

After the 24-hour incubation, the media was removed and wells were washed three times with PBS to remove extracellular nanoparticles or dye. Following the final wash, 50 µL of PBS was added to each well as a readout buffer.

Absorbance values were measured using a plate reader. FA absorbance was measured at 350 nm to evaluate folate receptor mediated targeting. Cy5 fluorescence was measured by setting excitation wavelength at 646 nm, emission wavelength at 662 nm, and a 7 nm bandwidth to assess fluorescent intensity.

### IONP Characterization

PEG-FA-Cy5-IONPs Characterization:

### UV-Vis Spectroscopy

Ultraviolet-visible (UV-Vis) spectroscopy was used to assess the optical characteristics of the synthesized nanoparticles. Aliquots of PEG-FA-Cy5-IONPs, IONP-only controls, Cy5-only controls, and folic acid-only controls were used in 24 and 96 well plates, and absorbance spectra was recorded using a microplate reader across the relevant wavelength range. UV-Vis was used to confirm the absorbance characteristics of the Cy5 chromophore and the folic acid, generating a standard curve, and to evaluate successful surface functionalization to the nanoparticle.

Standard curves for folic acid (using absorption value of 350 nm) and Cy5 (using excitation wavelength at 646 nm, emission wavelength at 662 nm, and a 7 nm bandwidth) were generated to allow for analysis of surface modification and conjugation efficiency.

A FA standard curve 5050 (FIG. 5) was created to quantify the amount of FA retained on PEG-FA-IONPs following purification. To avoid overflow values from highly concentrated samples, both folic acid and PEG-FA-IONP stocks were diluted to matching concentrations prior to measurement. Triplicate wells in a 24 well plate were prepared for each dilution to ensure reproducibility. Samples were prepared at final concentrations of 0.03 mg/mL, 0.02 mg/mL, and 0.01 mg/mL, along with a 1 mL HPLC grade ultrapure water blank used as the baseline reference.

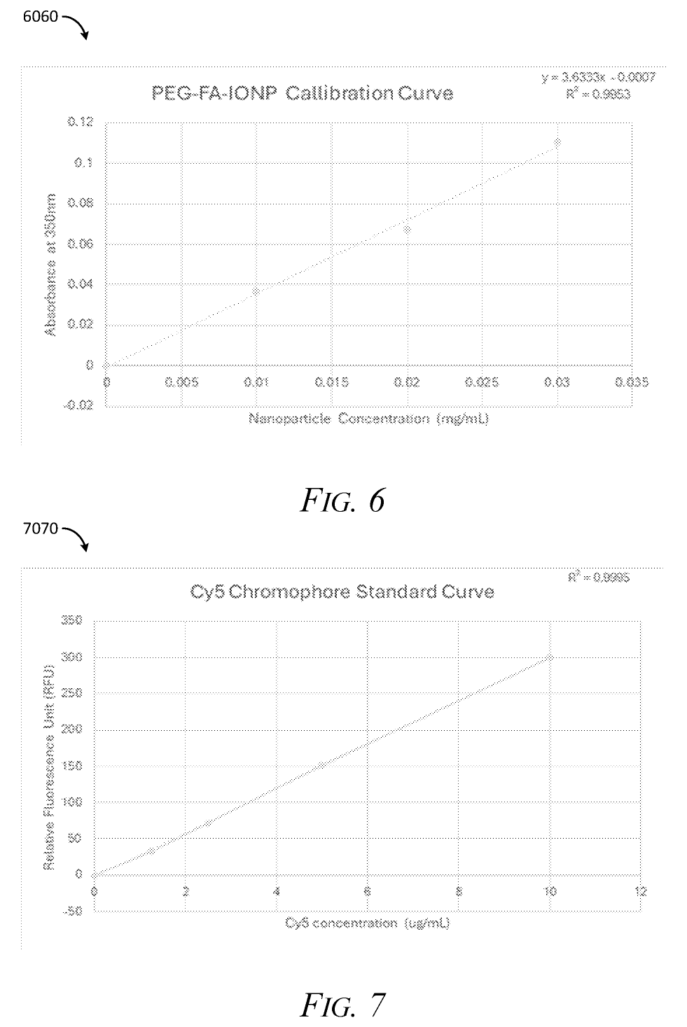

Absorbance was measured at 350 nm, a strong absorption peak of folic acid. The resulting FA known concentrations generated a linear calibration curve 6060 (FIG. 6), with an R² value of 0.9999, confirming linear correlation across the tested known concentration range. PEG-FA-IONP samples also generated a concentration-dependent absorbance response, yielding an R² value of 0.9953, following Beer-Lambert behavior within the measured range.

Using the folic acid calibration curve 5050, the FA concentration in each PEG-FA-IONP dilution was calculated by rearranging the slope-intercept equation of the standard curve (y= mx + b) to solve for concentration (x = (y – b)/m). The resulting FA concentrations were then multiplied by the corresponding dilution factors to obtain the FA concentration in the original 1 mg/mL PEG-FA-IONP stock. This value was then scaled to the total dried batch mass to determine the amount of folic acid retained after purification. From this calculation, the PEG-IONP-FA formulation was determined to contain 5.66 mg of folic acid per batch, corresponding to a 28.3 % conjugation efficiency relative to the 20 mg of folic acid added to the alkaline solution to create the coating base.

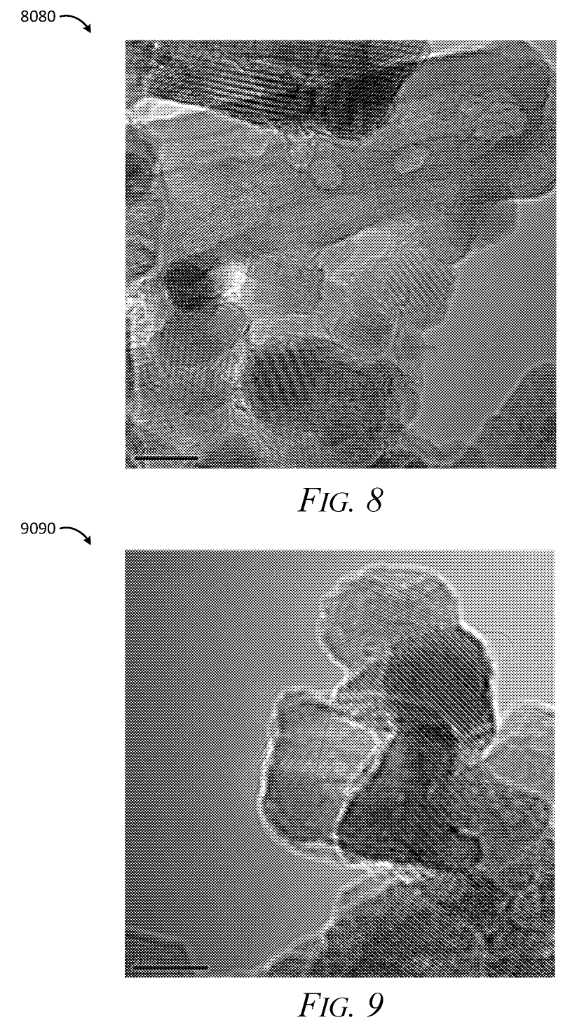

A Cy5 standard curve 7070 (FIG. 7) was generated to verify successful dye conjugation to the PEG-FA-IONP formulation. Cy5 fluorescence was measured by setting excitation wavelength at 646 nm, emission wavelength at 662 nm, and a 7 nm bandwidth to assess fluorescent intensity. The standard curve was prepared in a 24 well plate using 1.25 µg/mL, 2.5 µg/mL, 5 µg/mL, and 10 µg/mL Cy5 solutions in HPLC grade ultrapure water, producing a linear concentration-dependent emission response across the tested range with an R² value of 0.9995. Performing this standard curve first allows us to evaluate any potential self-quenching effects at higher Cy5 concentrations and identify a mid-range condition that maintained fluorescent emission without signal suppression. Based on this analysis, the 2.5 µg/mL condition was selected for all subsequent characterization experiments.

At the selected 2.5 µg/mL condition, the PEG-FA-Cy5-IONP samples exhibited a fluorescence intensity approximately 2.6 times than the free Cy5 from the standard curve. This enhancement can be attributed to two mechanisms. First, free Cy5 molecules undergo fluorescence self-quenching due to unrestricted freedom and free dye-dye interactions with the solution, whereas Cy5 molecules conjugated to the IONP surface are spatially separated from one another because of nanoparticle-nanoparticle electrostatic repulsion, reducing the fluorescence quenching effect. Second, metal-oxide nanoparticles alter the local electromagnetic field and dielectric environment, which can increase radiative decay rates and enhance the emissions efficiency of surface-bound chromophores. These two mechanisms explain why conjugated Cy5 on PEG-FA-Cy5-IONPs produced higher fluorescent emission than free dye at the same concentration

### Transmission Electron Microscopy TEM

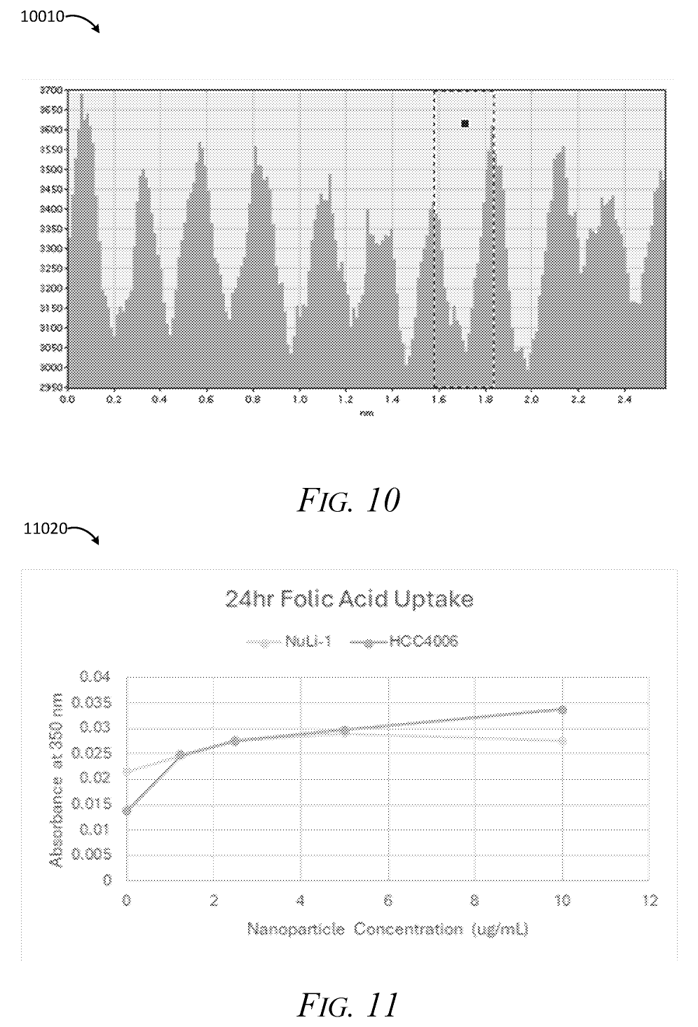

Transmission electron microscopy (TEM) was used to evaluate the morphology and core size of the synthesized NPs. One drop of the nanoparticle suspension was deposited onto a copper grid with a carbon coating and allowed to air dry at room temperature for 30 minutes. Excess liquid was carefully removed using filter paper to ensure that an even distribution of nanoparticles was on the grid surface. TEM analysis provided high-resolution information regarding the structure of the nanoparticles, including particle shape and core diameter. Two nanoparticle formulations were imaged: one prepared 24 hours prior to depositing on the TEM grid 8080 (FIG. 8), and one prepared 28 days hours prior to depositing on the TEM grid 9090 (FIG. 9). This comparison allowed assessment of both the immediate structural characteristics of the synthesized nanoparticles, as well as their long-term stability following storage.

For both samples, the nanoparticles exhibited spherical morphology and appeared tightly clustered, consistent with the expected behavior of magnetite-based iron oxide cores. Individual cores that were clearly distinguishable were analyzed for size analysis. The 24-hour sample displayed an average core diameter of 4.95 nm, whereas the 28-day sample displayed a larger average diameter of 8.91 nm.

Line analysis 10010 of the TEM images revealed distinct lattice fringes with a measured spacing of 2.5 angstrom, measured as the distance between adjacent intensity peaks as shown in FIG. 10. This spacing corresponds to the (311) crystal plane of magnetite. In the magnetite peak list, the (311) reflection carries an intensity of I % = 100, indicating it is the strongest and most characteristic peak of magnetite, confirming the identity of the nanoparticle core.

### Dynamic Light Scattering and Zeta Potential

Dynamic light scattering (DLS) and zeta potential analysis were used to characterize the hydrodynamic diameter, polydispersity, and surface charge of the synthesized nanoparticles. Nanoparticle suspensions of 1 mg/mL were loaded into glass cuvettes for DLS measurements or folded capillary cells for zeta potential analysis. Measurements were performed at room temperature, and each sample was analyzed in triplicates to ensure reproducibility. Zeta potential measurements were conducted to determine the surface charge and colloidal stability of the three nanoparticle formulations. Zeta potential provides insight into the electrostatic repulsion behavior between particles, with higher values (typically > ± 30 mV) indicating improved dispersion stability and reduced aggregation.

DLS provided the hydrodynamic diameter and polydispersity index (PDI), which details the width of the particle size distribution. A low PDI, typically < 0.2, indicates a uniform and well dispersed nanoparticle population while a higher PDI may suggest aggregation. Maintaining a low PDI is essential for ensuring consistent and reproducible biological behavior, as well as enhancing uptake and stability.

The IONP-only formulation displayed an average hydrodynamic diameter of 211.6 nm, while the PEG-FA-IONP sample showed a slight increase to 220.8 nm, reflecting the addition of the polymer-ligand (PEG-FA) coating. The PEG-FA-Cy5-IONP formulation displayed a larger hydrodynamic diameter of 331.6 nm. Typically, surface functionalized IONPs display hydrodynamic diameters in the ca. 400 nm range. The results we have obtained therefore fall within the acceptable size range for coated iron-oxide nanoparticles, with the progressive increase in hydrodynamic diameter across the three formulations corresponding with each successful surface modification step.

The polydispersity index (PDI) was evaluated for all three nanoparticle formulations to assess uniform particle size distributions and determine how much aggregation is occurring within each sample. PDI values closer to 0 indicate a more monodisperse population, whereas values greater than 0.3 indicate a broader distribution or potential aggregation. The IONP-only formulation displayed a PDI of 0.349, indicating a moderate polydisperse suspension consistent with partially aggregated iron-oxide nanoparticles. This could be a result of the formulation lacking any stabilizing reagent such as PEG. Following PEGylation and folic-acid conjugation, the PEG-FA-IONP formulation displayed a similar PDI of 0.358, suggesting that while the polymer-ligand coating did increase hydrodynamic diameter, it did not substantially enhance the dispersion of the nanoparticles in solution. In contrast, the PEG-FA-Cy5-IONP formulation demonstrated a significantly lower PDI of 0.097, indicating a highly uniform and monodisperse nanoparticle population in suspension. This improvement suggests that the Cy5 conjugation enhances colloidal stability, likely through increasing surface charge and therefore steric repulsion, reducing aggregation. The low PDI of < 0.1 is a characteristic of well stabilized nanoparticle formulations and supports the suitability of PEG-FA-Cy5-IONPs for biological applications.

The IONP-only formulation displayed a zeta potential of -7.73 mV, indicating a weakly negative surface charge. This low charge suggests minimal electrostatic repulsion which corresponds with the aggregation observed in the PDI results. Following PEGylation and folic acid conjugation, the PEG-FA-IONP formulation displayed a more negative zeta potential of -15.4 mV. This shift in a more negative charge is likely resulting from the negatively charged PEG coating, providing improvements in colloidal stability which it is known for. The PEG-FA-Cy5-IONP formulation demonstrated the most drastic change, with a zeta potential of -31.8 mV. This substantial increase in negative surface charge is consistent with the conjugation of sulf-Cy5-NHS, which carries a negative charge due to the NHS ester group. This contribution enhances electrostatic repulsion between particles, displaying a remarkably low 0.097 PDI. The PEG-FA-Cy5-IONP formulation exhibits the most favorable electrostatic profile, supporting its used for biological applications.

### MTT Cytotoxicity Assay and Cellular Uptake

Two human cell lines were used in this study to evaluate nanoparticle uptake and cytotoxicity: NuLi-1 and HCC4006. NuLi-1 cells (normal human bronchial epithelial; normal folate receptor count) serve as a non-cancerous control cell line model to determine baseline nanoparticle interactions in healthy epithelial tissue. The HCC4006 cells (human lung adenocarcinoma; overexpressed folate receptor count) were used as the targeted cancer cell model due to their elevated folate receptor expression. Both cell lines were cultured in an incubator (37 °C, 5 % CO₂) and maintained in their recommended growth media.

The MTT assay was used to evaluate metabolic activity and viability of cells following nanoparticle exposure. The assay used the ability of mitochondria enzymes to reduce the yellow MTT salt into an insoluble purple formazan crystal. This reduction only occurs in metabolically active cells, relating formazan production to overall cell viability. Absorbance was measured at 590 nm due to the strong optical signal formazan exhibits. Higher absorbance values would correlate to greater metabolic activity and therefore higher cell viability.

Nanoparticle uptake was analyzed using two absorbance signals. First, absorbance at 350 nm was used to detect the characteristic folic acid peak, providing an assessment of targeted delivery through the folate receptors. Second, Cy5 fluorescence intensity was measured using the manufacturer recommended excitation wavelength of 646 nm and emission wavelength of 662 nm, providing a complementary optical signal associated with the Cy5 chromophore.

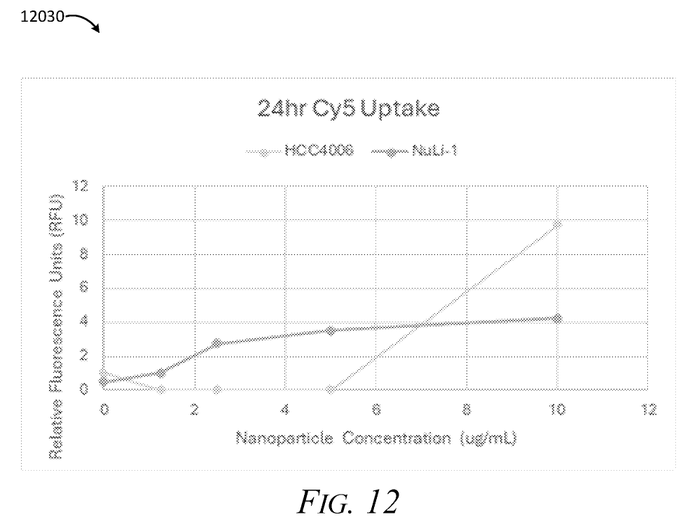

Folic Acid absorbance measurements demonstrated a dose-dependent increase in uptake for HCC4006 cells 11020 (FIG. 11). As nanoparticle concentration increased, HCC4006 displayed a strong positive trajectory, consistent with active folate receptor targeting. In contrast, NuLi-1 cells exhibited a minimal rise in absorbance values at low nanoparticle concentrations, followed by a decline at higher doses. This pattern aligns with their low folate receptor expression and suggests limited receptor mediated targeting.

Cy5 fluorescence intensity 12030 (FIG. 12) displayed a threshold response in HCC4006. Minimal or no detectable Cy5 signal was observed at concentrations below 10 µg/mL, followed by a sharp increase at the highest dose. A possible explanation is that HCC4006 cells internalize nanoparticles so efficiently at lower doses that the total Cy5 signal remains below the detection limit of the assay until a high extracellular concentration is reached. Once this threshold is exceeded, Cy5 fluorescence becomes measurable and produces the sharp increased observed at the highest dose. In contrast, NuLi-1 cells showed a gradual increase in Cy5 fluorescence across all doses. Notably, at the highest concentration the fluorescence intensity in HCC4006 cells was ca. 2.3 fold that of NuLi-1, indicative of greater overall nanoparticle uptake despite the absence of detectable signal at intermediate doses.

Throughout this application, various publications are referenced. The disclosures of these publications in their entireties are hereby incorporated by reference into this application to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for providing a tumor discovering compound, comprising:
synthesizing iron oxide (Fe₃O₄) nanoparticles (IONPs);
coating the IONPs with a poly(ethylene glycol) (PEG) layer, wherein the PEG layer comprises a folic acid component (PEG-FA) to provide a PEG-FA coated IONP (PEG-FA-IONP);
purifying the PEG-FA-IONPs;
labeling the PEG-FA-IONPs with a fluorescent chromophore to provide a PEG-FA-dye-IONP; and
purifying the PEG-FA-dye-IONPs.

2. The method of claim 1, wherein synthesizing the IONPs comprises:
preparing an alkaline solution in a first reaction vessel;
preparing an iron salt precursor solution in a second reaction vessel;
maintaining the iron salt precursor solution at a temperature of from 60 °C to 100 °C, a pH of from 8 to 10, and stirring at a rate of from 300 revolutions per minute (RPM) to 400 RPM in the second reaction vessel; and
adding the alkaline solution dropwise to the iron salt precursor solution in the second reaction vessel.

3. The method of claim 2, wherein coating the IONPs with the PEG-FA layer comprises:
preparing a PEG-FA solution in a third reaction vessel;
maintaining the iron salt precursor solution at a temperature of from 60 °C to 100 °C, a pH of from 8 to 10, and stirring at a rate of from 300 revolutions per minute (RPM) to 400 RPM in the second reaction vessel; and
adding the PEG-FA solution dropwise to the iron salt precursor solution in the second reaction vessel.

4. The method of claim 3, wherein purifying the PEG-FA-IONPs comprises;
magnetically separating the PEG-FA-IONPs from a reaction medium comprising unreacted salts or excess reagents;
washing the magnetically separated PEG-FA-IONPs; and
drying the PEG-FA-IONPs.

5. The method of claim 4, wherein labeling the PEG-FA-IONPs with a fluorescent chromophore to provide the PEG-FA-dye-IONPs comprises:
adding the PEG-FA-IONPs to a phosphate-buffered saline (PBS) solution to provide a PEG-FA-IONPs/PBS solution;
adding an ester-functionalized dye solution to the PEG-FA-IONPs/PBS solution to provide a dye-PEG-FA-IONPs solution; and
incubating the dye-PEG-FA-IONPs solution to provide the PEG-FA-dye-IONPs.

6. The method of claim 5, wherein purifying the PEG-FA-dye-IONPs comprises:
magnetically separating the PEG-FA-dye-IONPs from a supernatant;
washing the separated PEG-FA-dye-IONPs in a PBS solution; and
washing the separated PEG-FA-dye-IONPs in deionized water.

7. A method of identifying cancerous cells in a body, comprising:
introducing a fluorescent chromophore-labeled-cancer-specific-ligand-functionalized nanoparticle into the body;
binding the fluorescent chromophore-labeled-cancer-specific-ligand-functionalized nanoparticle to a cell overexpressing folate receptors; and
imaging the cells overexpressing folate receptors having the fluorescent chromophore-labeled-cancer-specific-ligand-functionalized nanoparticle bound thereon,
wherein imaging comprises contrast-enhanced magnetic resonance imaging (MRI), optical imaging, or a combination thereof to specify a tumor cell.

8. The method of claim 7, wherein the nanoparticle comprises an iron oxide (Fe₃O₄) nanoparticle (IONP).

9. The method of claim 7, wherein the cancer-specific ligand comprises folic acid.

10. The method of claim 7, wherein the fluorescent chromophore comprises a near-infrared (NIR) light emitting dye.

11. The method of claim 7, wherein the fluorescent chromophore comprises a visible light emitting dye.

12. The method of claim 7, wherein the fluorescent chromophore-labeled-cancer-specific-ligand-functionalized nanoparticle comprises a fluorescent dye-labeled, poly(ethylene glycol)-folic acid (PEG-FA) coated iron oxide nanoparticle (PEG-FA-dye-IONP).

13. The method of claim 12, wherein introducing the fluorescent chromophore-labeled-cancer-specific-ligand-functionalized nanoparticle into the body comprises injecting a dispersion of the PEG-FA-dye-IONPs into the body.

14. The method of claim 13, wherein binding the PEG-FA-dye-IONPs to a cell overexpressing folate receptors comprises urging the PEG-FA-dye-IONPs toward a cell.

15. The method of claim 14, wherein imaging the PEG-FA-dye-IONPs provides a T₂-weighted MRI contrast indicated by dark areas in an MRI image.

16. The method of claim 15, wherein imaging the PEG-FA-dye-IONPs comprises optical imaging, the optical imaging comprising exciting the fluorescent chromophore to provide an optical image of the PEG-FA-dye-IONPs bound to the cell overexpressing folate receptors.

17. The method of claim 16, wherein the fluorescent chromophore comprises an excitement wavelength (λ) from 590 nanometers (nm) to 675 nm, an emission wavelength from 650 nm to 750 nm, and a full-width half-maximum (FWHM) from 5 nm to 50 nm.

18. The method of claim 17, wherein combining contrast-enhanced MRI and optical imaging provides an in-vitro and in-vivo safe cancer-cell identifying system.

19. A dual-modal tumor imaging system, comprising:
an iron oxide (Fe₃O₄) nanoparticle (IONP);
a poly(ethylene glycol) (PEG) layer disposed on the IONP, wherein the PEG layer comprises a folic acid component (PEG-FA); and
a fluorescent chromophore conjugated onto a portion of the folic acid component.

20. The dual-modal tumor imaging system of claim 19, wherein the fluorescent chromophore comprises an excitement wavelength (λ) from 590 nanometers (nm) to 675 nm, an emission wavelength from 650 nm to 750 nm, and a full-width half-maximum (FWHM) from 5 nm to 50 nm.
